(19) United States
(12) Patent Application Publication
(10) Pub. No.: US 2007/0142743 A1
(43) Pub. Date: Jun. 21, 2007

(54) Tissue sample needle actuator system and apparatus and method of using same

(76) Inventors: Kevin M. Provencher, Framingham, MA (US); Keith Orr, Boston, MA (US)

Correspondence Address: BROMBERG & SUNSTEIN LLP, 125 SUMMER STREET, BOSTON, MA, 02110-1618, US

(21) Appl. No.: 11/305,302

(22) Filed: Dec. 16, 2005

(51) Int. Cl. A61B 10/00

(52) U.S. Cl. 600/562; 600/564

(57) ABSTRACT

A tissue sample needle actuator system and apparatus includes an actuation mechanism for, among other things, automatically thrusting a tissue sample needle forward and/or automatically rotating an inner tube of the tissue sample needle relative to an outer tube of the tissue sample needle to operate an integral snare. The actuation mechanism may perform other functions such as, for example, rotating the entire needle (e.g., during thrusting, during operation of the snare, or during a separate phase), which may, in some situations, facilitate insertion of the needle and/or obtaining a tissue sample. The actuator may also support a stylet for facilitating insertion of the needle and/or ejection of the tissue sample.

### FIELD OF THE INVENTION

The present invention relates to devices and methods for the removal of tissue samples from a body organ.

### BACKGROUND OF THE INVENTION

In the practice of medical diagnostics, it is often necessary to perform a biopsy to remove sample of the patient's tissue for pathological study. Various devices are known in the art that are intended to make the biopsy procedure faster, more reliable, easier to perform and reduces the chance of discomfort or injury to the patient.

Certain biopsy needle devices are disclosed in the following United States patents, all of which are hereby incorporated herein by reference in their entireties: U.S. Pat. No. 5,522,398 issued on Jun. 4, 1996 to Goldenberg et al. and assigned to Medsol Corporation; U.S. Pat. No. 5,634,473 issued on Jun. 3, 1997 to Goldenberg et al. and assigned to Medsol Corporation; U.S. Pat. No. 5,843,001 issued on Dec. 1, 1998 to Goldenberg; U.S. Pat. No. 6,015,391 issued on Jan. 18, 2000 to Rishton et al.; U.S. Pat. No. 6,033,369 issued on Mar. 7, 2000 to Goldenberg; and U.S. Pat. No. 6,340,351 issued on Jan. 22, 2002 to Goldenberg. These devices have a needle comprised of an outer cannula and an inner tube with an integral snare having a coil. The inner tube is attached at its distal end to the outer cannula. Proximal to the attached portion of the inner tube is a coil. After insertion of the needle into a patient's bone with the aid of a stylet, a handle is used to rotate the inner tube relative to the outer cannula and effects a decrease in diameter of the coil portion, thereby constricting the tissue so that the tissue is restricted to cause a portion of the tissue to remain in the inner tube during withdrawal of the device. The constricting action of such a device does not necessarily cut the tissue since the snare does constrict to zero diameter, but rather the tissue is often ripped upon rearward motion of the needle. The ripping action is more efficacious for bone marrow than for soft tissue, which tends to be more elastic. For example, such a device would generally be unreliable in extracting soft tissue such as that which might be extracted in a breast biopsy.

### SUMMARY OF THE INVENTION

In accordance with one aspect of the invention there is provided an actuator for operating a tissue sample needle. The actuator includes a housing and an actuation mechanism disposed within the housing for operating the tissue sample needle, which involves at least automatically rotating an inner tube of the tissue sample needle relative to an outer tube of the tissue sample needle to operate an integral snare.

In accordance with another aspect of the invention there is provided a tissue sample actuation system comprising a tissue sample needle actuator and a tissue sample needle installed in the actuator, wherein the tissue sample needle includes an outer tube and an inner tube having an integral snare such that rotation of the inner tube relative to the outer tube operates the snare, and wherein the actuator includes an actuation mechanism disposed within a housing for operating the tissue sample needle, which involves at least automatically rotating the inner tube relative to the outer tube to operate the integral snare.

In related embodiments, the actuation mechanism may automatically thrust the tissue sample needle prior to automatically rotating the inner tube relative to the outer tube. Automatic thrusting of the needle may be performed in a first phase of actuation and automatic rotation of the inner tube relative to the outer tube may be performed in a second subsequent phase of actuation. At least one of the housing and the actuation mechanism may include a stopping mechanism for stopping thrusting of the needle while allowing the subsequent rotation of the inner tube relative to the outer tube. The actuation mechanism may rotate the inner tube while constraining the outer tube for automatically rotating the inner tube relative to the outer tube. Alternatively, the actuation mechanism may rotate the outer tube while constraining the inner tube for automatically rotating the inner tube relative to the outer tube.

In further related embodiments, the actuation mechanism may include a movable sleeve for automatically thrusting the tissue sample needle and/or automatically rotating the inner tube relative to the outer tube. The sleeve may control both the automatic thrusting of the tissue sample needle and the automatic rotation of the inner tube relative to the outer tube. The tissue sample needle may be disposed within the sleeve. The actuation mechanism may rotate the inner tube while constraining the outer tube for automatically rotating the inner tube relative to the outer tube, in which case the sleeve may include a slot for constraining rotational movement of the outer tube and a channel for driving rotational movement of the inner tube during linear motion of the sleeve. The actuation mechanism may include at least one of a spring, a plurality of springs, an electric motor, pneumatics, and hydraulics for driving movement of the sleeve.

In still further related embodiments, the actuation mechanism may include a cocked position in which the needle is prepared for operation. The actuation mechanism may include a cocking mechanism for placing the actuation mechanism in the cocked position, such as a handle for manually cocking the actuation mechanism or at least one of an electric motor, pneumatics, and hydraulics for driving the cocking mechanism. The actuation mechanism may include a trigger for selectively maintaining the actuation mechanism in the cocked position and releasing the actuation mechanism from the cocked position. A stylet may be included such that the stylet protrudes from a distal end of the needle prior to thrusting and retracts from the distal end of the needle during thrusting.

In accordance with another aspect of the invention there is provided a method for obtaining a tissue sample. The method involves cocking a tissue sample needle actuation system including a tissue sample needle installed in a tissue sample actuator, inserting the tissue sample needle into a body part, and firing the tissue sample needle actuation system to operate the tissue sample needle, which involves at least automatically rotating an inner tube of the tissue sample needle relative to an outer tube of the tissue sample needle to operate an integral snare.

In related embodiments, the actuator may automatically thrust the tissue sample needle prior to automatically rotating the inner tube relative to the outer tube. The actuator may rotate the inner tube while constraining the outer tube for automatically rotating the inner tube relative to the outer tube. Alternatively, the actuator may rotate the outer tube while constraining the inner tube for automatically rotating the inner tube relative to the outer tube. The method may further involve withdrawing the tissue sample needle from the body part and ejecting a tissue sample from the tissue sample needle.

### BRIEF DESCRIPTION OF THE DRAWINGS

The foregoing and advantages of the invention will be appreciated more fully from the following further description thereof with reference to the accompanying drawings wherein:

FIGS. 1-12 show the relevant components for an 11-gauge tissue sample needle, in accordance with a first exemplary embodiment of the present invention, wherein:

FIGS. 1A-1E are schematic diagrams showing an inner tube assembly 100 for an 11-gauge tissue sample needle, in accordance with an exemplary embodiment of the present invention;

FIG. 2 is a schematic diagram showing the snare 104 in greater detail;

FIGS. 3A-3C are schematic diagrams showing the inner tube housing 106 in greater detail;

FIG. 4 is a schematic diagram showing an outer tube assembly 400 for an 11-gauge tissue sample needle, in accordance with an exemplary embodiment of the present invention;

FIGS. 5A-5C are schematic diagrams showing the outer tube 402 in greater detail;

FIGS. 6A-6C are schematic diagrams showing the outer tube housing 406 in greater detail;

FIGS. 7A-7B are schematic diagrams showing a needle assembly 700 in accordance with an exemplary embodiment of the present invention;

FIG. 8 is a schematic diagram showing the distal end 702 of the needle assembly 700 in greater detail;

FIG. 9 is a schematic diagram showing the needle assembly 700 after grinding;

FIG. 10 is a schematic diagram showing the point configuration 902 in greater detail;

FIG. 11 is a schematic diagram showing a stylet assembly 1100 for an 11-gauge tissue sample needle in accordance with an exemplary embodiment of the present invention; and

FIGS. 12A-12D are schematic diagrams showing the stylet housing 1106 in greater detail;

FIGS. 13-24 show the relevant components for a 14-gauge tissue sample needle, in accordance with a second exemplary embodiment of the present invention, wherein:

FIGS. 13A-13D are schematic diagrams showing an inner tube assembly 1300 for a 14-gauge tissue sample needle, in accordance with an exemplary embodiment of the present invention;

FIG. 14 is a schematic diagram showing the snare 1304 in greater detail;

FIGS. 15A-15C are schematic diagrams showing the inner tube housing 1306 in greater detail;

FIG. 16 is a schematic diagram showing an outer tube assembly 1600 for a 14-gauge tissue sample needle, in accordance with an exemplary embodiment of the present invention;

FIGS. 17A-17B are schematic diagrams showing the outer tube 1602 in greater detail;

FIGS. 18A-18C are schematic diagrams showing the outer tube housing 1606 in greater detail;

FIGS. 19A-19B are schematic diagrams showing a needle assembly 1900 in accordance with an exemplary embodiment of the present invention;

FIG. 20 is a schematic diagram showing the distal end 1902 of the needle assembly 1900 in greater detail;

FIG. 21 is a schematic diagram showing the needle assembly 1900 after grinding;

FIG. 22 is a schematic diagram showing the point configuration 2002 in greater detail;

FIG. 23 is a schematic diagram showing a stylet assembly 2300 for a 14-gauge tissue sample needle in accordance with an exemplary embodiment of the present invention; and

FIGS. 24A-24D are schematic diagrams showing the stylet housing 2306 in greater detail;

FIG. 25 shows some exemplary alternative snare configurations, wherein:

FIG. 25A shows an enlarged view of an exemplary snare for a 14-gauge needle having two deformable members;

FIG. 25B shows an alternative snare configuration with two deformable members, each forming a single coil;

FIG. 25C shows an alternative snare configuration with two deformable members, each forming a double coil;

FIG. 25D shows an alternative snare configuration with six deformable members; and

FIG. 25E shows an alternative snare configuration with seven deformable members; and

FIGS. 26-35 show the relevant components for an actuator and an actuation system, in accordance with an exemplary embodiment of the present invention, wherein:

FIGS. 26A-26B are schematic diagrams showing an actuator 2500 in accordance with an exemplary embodiment of the present invention;

FIG. 27 is a schematic diagram showing an actuation system 2700 in accordance with an exemplary embodiment of the present invention;

FIG. 28 is a schematic diagram showing a top view of the actuation system 2700 with the top cover of the actuator housing 2502 removed so as to expose the inner workings and components of the actuation system 2700;

FIG. 29 is a schematic diagram showing a side view of the actuation system 2700;

FIG. 30 is a schematic diagram showing a perspective view of the actuating system 2700 in a fired position;

FIG. 31 is a schematic diagram showing the needle/sleeve assembly 3100 including needle assembly 700/1900 disposed within sleeve 2702;

FIG. 32 is a schematic diagram showing a top view of the bottom portion of the actuator housing 2502;

FIG. 33 is a schematic diagram showing a bottom view of the sleeve 2702;

FIG. 34 shows one piece of the sleeve 2702; and

FIG. 35 shows the other piece of the sleeve 2702.

### DETAILED DESCRIPTION OF SPECIFIC EMBODIMENTS

In embodiments of the present invention, a tissue sample needle actuator system and apparatus includes an actuation mechanism for, among other things, automatically thrusting a tissue sample needle forward and/or automatically rotating an inner tube of the tissue sample needle relative to an outer tube of the tissue sample needle to operate an integral snare. Embodiments of the present invention are not limited to any particular type of tissue sample needle or snare configuration, and so can be used with single-coil snares of the type discussed in the background above or with other types of snares described herein below. The actuation mechanism may perform other functions such as, for example, rotating the entire needle (e.g., during thrusting, during operation of the snare, or during a separate phase), which may, in some situations, facilitate insertion of the needle and/or obtaining a tissue sample. The actuator may also support a stylet for facilitating insertion of the needle and/or ejection of the tissue sample.

The actuator may be included as part of an overall actuation system that includes a tissue sample needle installed in or otherwise integral to the tissue sample needle actuator. In such an actuation system, the actuator may be configured to be disposable after a predetermined number of needle operations, or may be configured to be reusable by virtue of replaceable needles. The actuation system may optionally include a stylet to facilitate insertion of the needle into a body part and/or ejection of the tissue sample.

Exemplary embodiments are described below with reference to various tissue sample needles and related components as described in related U.S. patent application Ser. No. XX/XXX,XXX entitled TISSUE SAMPLE NEEDLE AND METHOD OF USING SAME, which was filed on even date herewith in the name of Kevin Provencher, and is hereby incorporated herein by reference in its entirety. These tissue sample needles include a snare designed to efficiently pinch or sever tissue or tissue-like material upon actuation of the snare with a twisting action. The snare is typically tubular in shape and may be integral to the inner tube of the tissue sample needle. The snare is effective on soft tissue as well as harder tissue such as bone marrow. Efficient pinching or severing of tissue is achieved by a deformable zone of the snare having two or more deformable members that deform inwardly upon actuation. Some or all of the deformable members may contact other deformable members upon twisting in one direction to effectively reduce the inner diameter of the snare to zero. Twisting in the opposite direction will typically expand the diameter of the tube and allow the tissue to be recovered. The deformable members may be helical or serpentine in shape. There may be two, three, four, five, six, seven, or even more deformable members. The deformable members may include sharp edges that are presented to the tissue upon actuation thereby increasing the chance of severing the tissue.

In embodiments, the twisting is achieved by directly or indirectly fixing the deformable members at a distal end to a support structure and at a proximal end to a tissue-holding structure. The zone of connection between the deformable member and the support structure may be rotationally offset from the zone of connection between the deformable member and the tissue-holding structure by a number of degrees. The offset may be advantageously be chosen to be related to the number of deformable members by an amount equal to 360° divided by the number of deformable members. The support structure serves to hold the distal end of the deformable members so that the members will deform upon application of a torsion via a rotation of the tissue-holding structure relative to the support structure. The support structure is typically an outer tube, but could be another structure, such as one or more rods that will serve to hold the distal end of the deformable members to allow the application of a rotational force.

The deformable member may be directly connected or attached to the support structure. For example, the deformable members may be flexible serpentine structures that are welded directly to a tubular support structure at their distal ends. Alternately, the deformable members may be indirectly connected or attached to the support structure. In embodiments discussed in more detail below, the deformable members may be flexible serpentine structures that are coupled to a cylindrical securing member and the cylindrical securing member may be coupled to the inside of an outer tubular support structure.

The tissue holding structure is typically a proximal region of a cylindrical support inner tube, but could be any structure capable of holding a tissue sample upon actuation of the snare. The tissue-holding structure is characterized by a lumen, an interior space in which a tissue sample is held.

In certain embodiments, the support structure is an outer tube and the snare is integral to an inner tube. The needle may be manufactured from metal (e.g., 304 stainless steel), plastic, or other material, and may advantageously be both sufficiently hard enough to cut or pinch tissue with rotation in one direction, yet resilient enough to allow release of the tissue upon rotation in the opposite direction. The needle may be designed to be disposable or reusable. The zone of deformation having the deformable members may be created by the removal of material from a zone of the inner tube to create fenestrations. The material removed may form in relief two or more helical or serpentine members. Removal of the material is readily accomplished by laser cutting but alternate modes of manufacture could be employed including wire electospark discharge machining (wire EDM), chemical or photochemical etching, or other suitable technique. The device may also be made by welding together cylindrical section of tubing and deformable members, although at potentially greater expense.

In use, the tissue sample needle is first inserted into a desired tissue, such as a suspected tumor. The needle is designed to work on soft tissues like kidney, liver, lung, thyroid, prostate or breast, but embodiments will also work on harder tissues like bone marrow. Insertion of the needle may be facilitated by the use of a stylet which is typically a sharpened rod retractably inserted through the center of the needle. The stylet may be retracted and the needle subsequently pushed in further to ensure the entry of tissue into the lumen of the tissue-holding structure. Alternately, the end of the needle may be sharp enough to penetrate without the use of a stylet. After insertion, the snare is actuated by rotation in a first direction of the tissue-holding structure relative to the support structure. Since the tissue holding structure is typically internal to the needle and the support structure external, it may be advantageous to hold the external structure fixed and rotate the internal structure and thereby avoid frictional resistance and potential tissue irritation that might result if the interior structure were held fixed and the exterior structure was rotated. However, in many cases, rotation of an external support structure will accomplish the same function. In either case, actuation of the device will cause an inward deformation of the deformable members and a resulting pinching or severing of the tissue in the zone of deformation.

The needle is then withdrawn. If the tissue is pinched or partially severed, the act of withdrawal may serve to rip the tissue, allowing it to remain in the lumen of the tissue-holding structure. If the snare is not closed tightly enough, a resilient soft tissue may slip through the snare, thereby remaining in the patient. If the tissue is cleanly severed, then no such ripping or slippage will occur, leading to more reliable operation that may be less injurious to the patient. Embodiments presented herein are designed to be more reliable by creating an increased constriction and an increased chance of complete or partial cutting.

After withdrawal, the sample is typically recovered for further diagnostic or forensic analysis such as a pathological analysis for the presence of cancerous cells by cytology, histology, immunohistochemistry, messenger RNA profiling or other technique. Recovery of the sample may be accomplished by rotation of the sample-holding structure relative to the support structure in a second direction opposite to the direction of the first rotation so as to expand the inner diameter of the snare. The sample is then typically extracted by applying a force to the sample to dislodge it from the sample-holding structure. The force is typically applied with a rod, which may be the stylet, but could also be, without limitation, a squeezing, decelerating, pneumatic or hydraulic force. The same needle may be used many times on the same patient. For example, 6, 8 or even more samples may be obtained using the same needle. When compared to the coiled snare design patented by Goldenberg as listed above, embodiments of the current invention employing multiple deformable members generally allow for pinching or cutting of the sample with a smaller degree or rotation of the inner and outer tubes, thus giving a greater reliability and confidence that the device will operate without malfunction to obtain the desired number of tissue samples. In contrast to embodiments of the present invention, needles of the Goldenberg coil design cannot effectively close to an internal diameter of zero and do not allow tissue to be severed without substantial ripping.

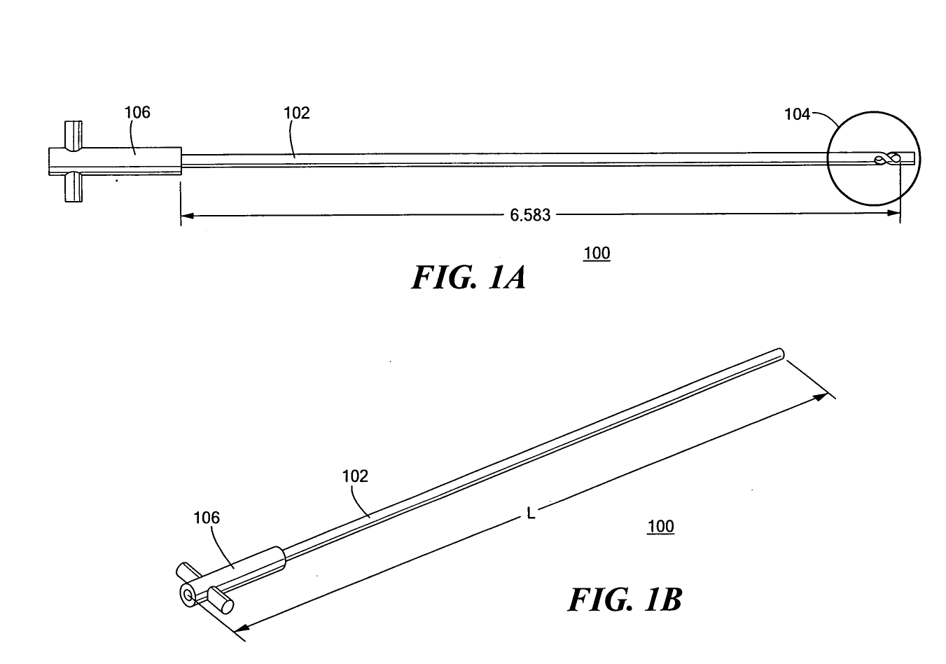

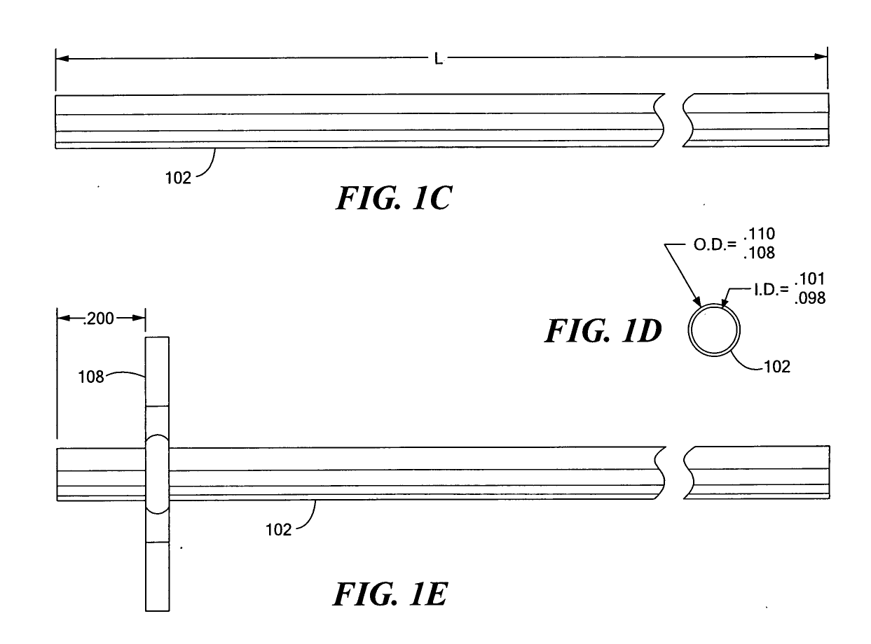

FIGS. 1-12 show the relevant components for an 11-gauge tissue sample needle, in accordance with a first exemplary embodiment of the present invention.

FIGS. 1A-1E are schematic diagrams showing an inner tube assembly 100 for an 11-gauge tissue sample needle, in accordance with an exemplary embodiment of the present invention. As shown in FIG. 1A, the inner tube assembly 100 includes an inner tube 102 with a snare 104 located at a distal end and an inner tube housing 106 located at a proximal end. FIG. 1B is a perspective view of the inner tube assembly 100. FIG. 1C is a side view of a blank inner tube 102. FIG. 1D is cross-sectional view of the inner tube 102 showing outer diameter between approximately 0.108 and 0.110 inches and an inner diameter between approximately 0.098 and 0.101 inches. FIG. 1E is a side view of the inner tube 102 showing a wire hub 108 that is formed on the inner tube 102 approximately 0.200 inches from the proximal end to help secure the inner tube housing 106 on the inner tube 102.

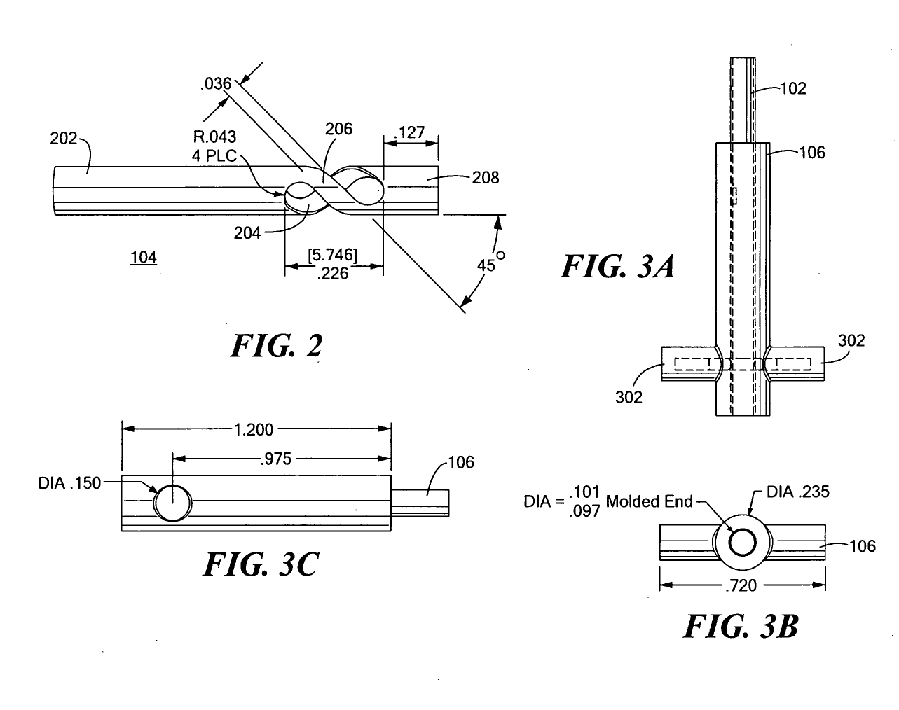

FIG. 2 is a schematic diagram showing the snare 104 in greater detail. The snare 104 includes two deformable members 204 and 206 that are coupled respectively at a proximal end to tube body 202 and at a distal end to a securing member 208. The deformable members 204 and 206 are serpentine/helical in shape and are approximately 0.036 inches in width and nominally oriented at an approximately 45 degree angle with respect to the axis of the inner tube 102. The deformable members 204 and 206 are typically formed by laser cutting the inner tube 102 to remove interstitial material.

FIGS. 3A-3C are schematic diagrams showing the inner tube housing 106 in greater detail. FIG. 3A is a top view of the inner tube housing 106. FIG. 3B is an end view of the inner tube housing 106. FIG. 3C is a side view of the inner tube housing 106. The inner tube housing 106 is typically molded onto the proximal end of the inner tube 102, although it could be produced in other ways. The inner tube housing 106 includes protrusions 302 (one on each side) approximately 0.150 inches in diameter. During use of the tissue sample needle, the protrusions 302 may be used for, among other things, preventing backward displacement and rotation of the needle during a thrusting of the needle and subsequently rotating the inner tube 102 relative to an outer tube during operation of the snare 104, as discussed below.

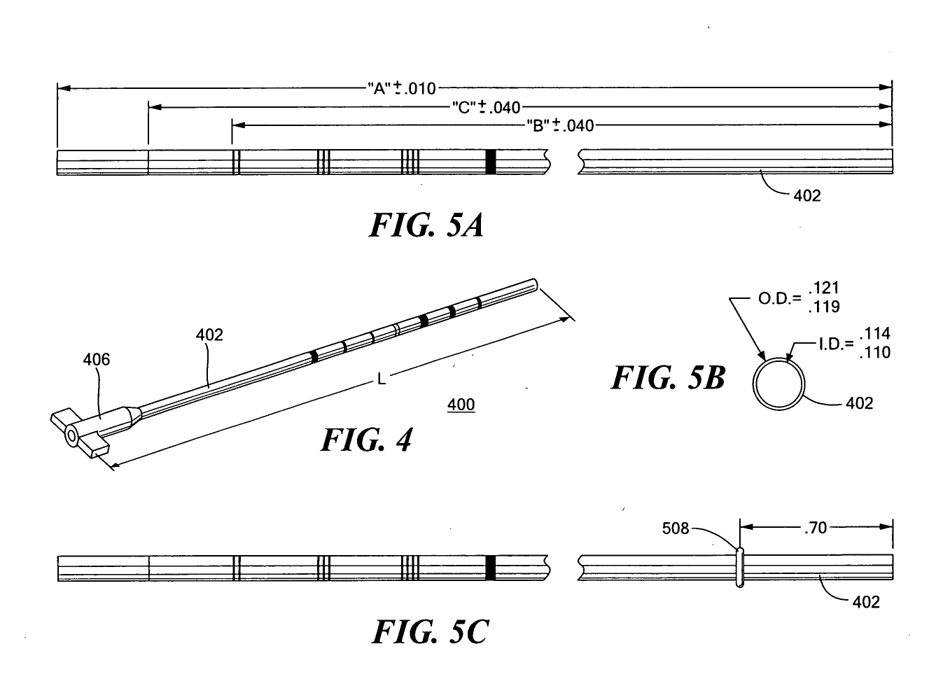

FIG. 4 is a schematic diagram showing an outer tube assembly 400 for an 11-gauge tissue sample needle, in accordance with an exemplary embodiment of the present invention. The outer tube assembly 400 includes an outer tube 402 with an outer tube housing 406 located at a proximal end. The outer tube 402 typically includes depth indicator markings toward the distal end to aid in obtaining a tissue sample.

FIGS. 5A-5C are schematic diagrams showing the outer tube 402 in greater detail. FIG. 5A is a side view of the outer tube 402 showing the depth indicator markings at approximately one centimeter intervals along a length of the outer tube 402. FIG. 5B is cross-sectional view of the outer tube 402 showing outer diameter between approximately 0.119 and 0.121 inches and an inner diameter between approximately 0.110 and 0.114 inches. FIG. 5C is a side view of the outer tube 402 showing a wire hub 508 that is formed on the outer tube 402 approximately 0.76 inches from the proximal end to help secure the outer tube housing 406 on the outer tube 402.

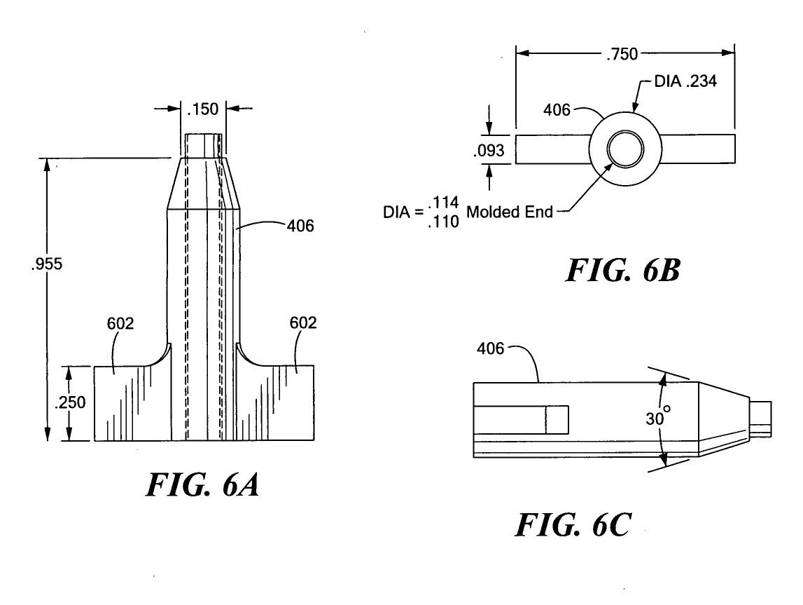

FIGS. 6A-6C are schematic diagrams showing the outer tube housing 406 in greater detail. FIG. 6A is a top view of the outer tube housing 406. FIG. 6B is an end view of the outer tube housing 406. FIG. 6C is a side view of the outer tube housing 406. The outer tube housing 406 is typically molded onto the proximal end of the outer tube 402, although it could be produced in other ways. The outer tube housing 406 includes protrusions 602 (one on each side). During use of the tissue sample needle, the protrusions 602 may be used for, among other things, maintaining linear movement of the needle during a thrusting of the needle and preventing rotation of the outer tube 402 during operation of the snare 104, as discussed below.

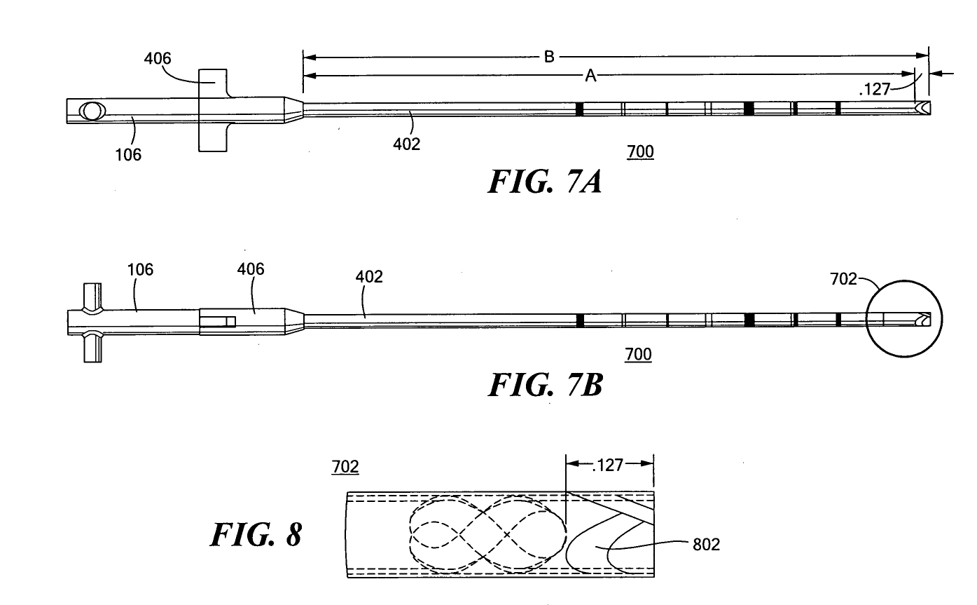

FIGS. 7A-7B are schematic diagrams showing a needle assembly 700 in accordance with an exemplary embodiment of the present invention. The needle assembly 700 includes an inner tube assembly 100 disposed within an outer tube assembly 400. The distal end of the inner tube 102 (and, more particularly, the securing member 208) is coupled to the distal end of the outer tube 402, for example, by laser welding about a circumference of the distal end of the outer tube 402. FIG. 7A is a side view of the needle assembly 700 showing the inner tube housing 106 protruding from the proximal end of the outer tube assembly 400. FIG. 7B is a top view of the needle assembly 700, highlighting the distal end 702 of the needle assembly 700 where the inner tube 102 and outer tube 402 are laser welded.

FIG. 8 is a schematic diagram showing the distal end 702 of the needle assembly 700 in greater detail. The inner tube 102 and outer tube 402 are laser welded in area 802, which is in the area of the securing member 208 and does not interfere with the deformable members 204 and 206. As discussed below, the distal end 702 of the needle assembly 700 is ground after welding in order to form a desired point configuration (e.g., a Franseen point). The weld is typically designed to substantially follow the outline of the grinding.

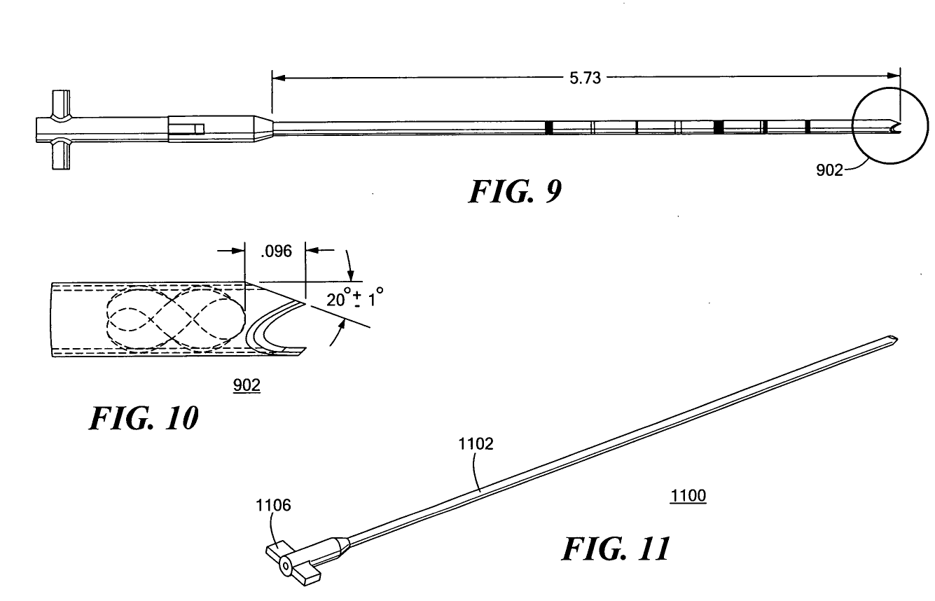

FIG. 9 is a schematic diagram showing the needle assembly 700 after grinding. As mentioned above, the distal end 702 of the needle assembly 700 is ground after welding in order to form a desired point configuration 902.

FIG. 10 is a schematic diagram showing the point configuration 902 in greater detail.

FIG. 11 is a schematic diagram showing a stylet assembly 1100 for an 11-gauge tissue sample needle in accordance with an exemplary embodiment of the present invention. The stylet assembly 1100 includes a stylet 1102 and a stylet housing 1106. The stylet 1102 is typically sharpened at its distal end. The stylet assembly 1100 may be disposed within the inner tube assembly 100 during operation of the tissue sample needle to facilitate insertion of the needle into a body tissue and/or to remove a tissue sample from the inner tube 102. The stylet assembly 1100 is typically designed so that the sharpened tip protrudes from the distal end of the needle when the stylet assembly 1100 if fully inserted through the inner tube assembly 100.

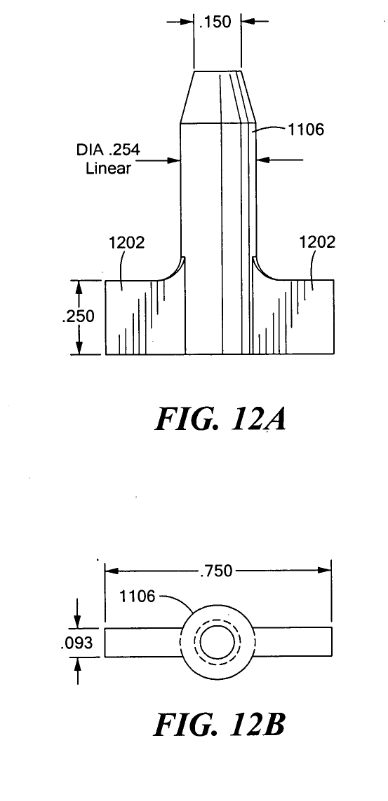

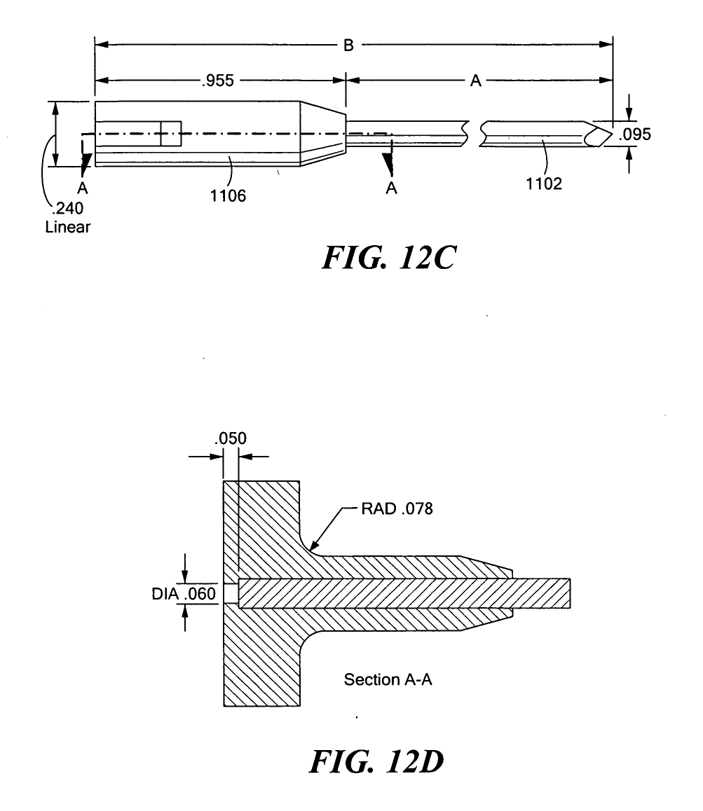

FIGS. 12A-12C are schematic diagrams showing the stylet housing 1106 in greater detail. FIG. 12A is a top view of the stylet housing 1106. FIG. 12B is an end view of the stylet housing 1106. FIG. 12C is a side view of the stylet housing 1106 showing the sharpened end. FIG. 12D is a cross-sectional view of the stylet housing 1106. The stylet housing 1106 is typically molded onto the proximal end of the stylet 1102, although it could be produced in other ways. The stylet housing 1106 includes protrusions 1202 (one on each side). During use of the tissue sample needle, the protrusions 1202 may be used for, among other things, securing the stylet assembly 1100, as discussed below.

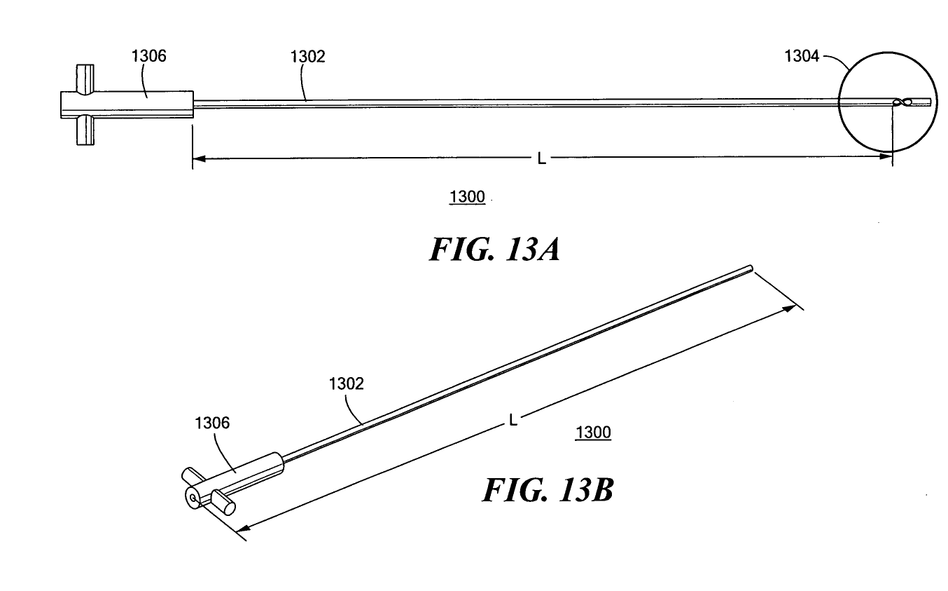

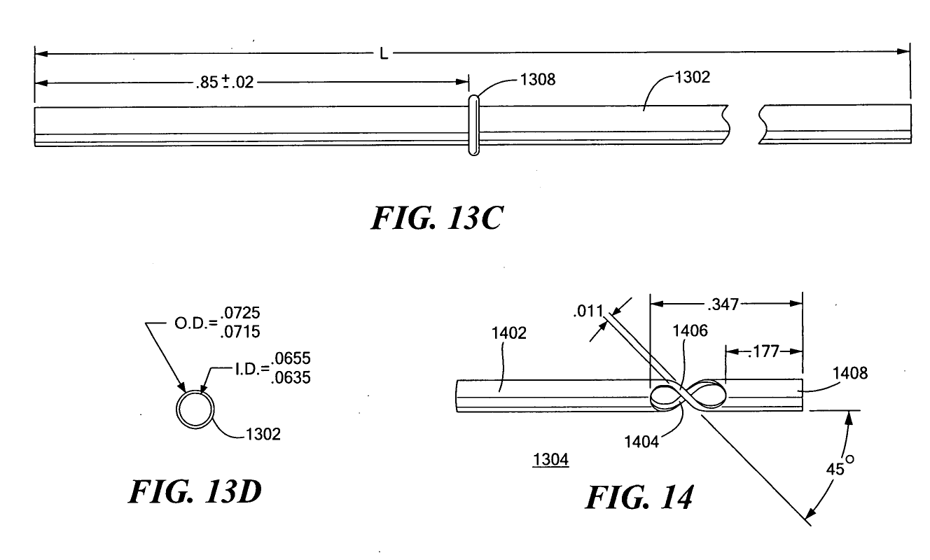

FIGS. 13-24 show the relevant components for a 14-gauge tissue sample needle, in accordance with a second exemplary embodiment of the present invention.

FIGS. 13A-13D are schematic diagrams showing an inner tube assembly 1300 for a 14-gauge tissue sample needle, in accordance with an exemplary embodiment of the present invention. As shown in FIG. 13A, the inner tube assembly 1300 includes an inner tube 1302 with a snare 1304 located at a distal end and an inner tube housing 1306 located at a proximal end. FIG. 13B is a perspective view of the inner tube assembly 1300. FIG. 13C is a side view of the inner tube 1302 showing a wire ring 1308 that is formed on the inner tube 1302 approximately 0.85 inches from the proximal end to help secure the inner tube housing 1306 on the inner tube 1302. FIG. 13D is cross-sectional view of the inner tube 1302 showing outer diameter between approximately 0.0715 and 0.0725 inches and an inner diameter between approximately 0.0635 and 0.0655 inches.

FIG. 14 is a schematic diagram showing the snare 1304 in greater detail. The snare 1304 includes two deformable members 1404 and 1406 that are coupled respectively at a proximal end to tube body 1402 and at a distal end to a securing member 1408. The deformable members 1404 and 1406 are serpentine/helical in shape and are approximately 0.011 inches in width and nominally oriented at an approximately 45 degree angle with respect to the axis of the inner tube 1302. The deformable members 1404 and 1406 are typically formed by laser cutting the inner tube 1302 to remove interstitial material.

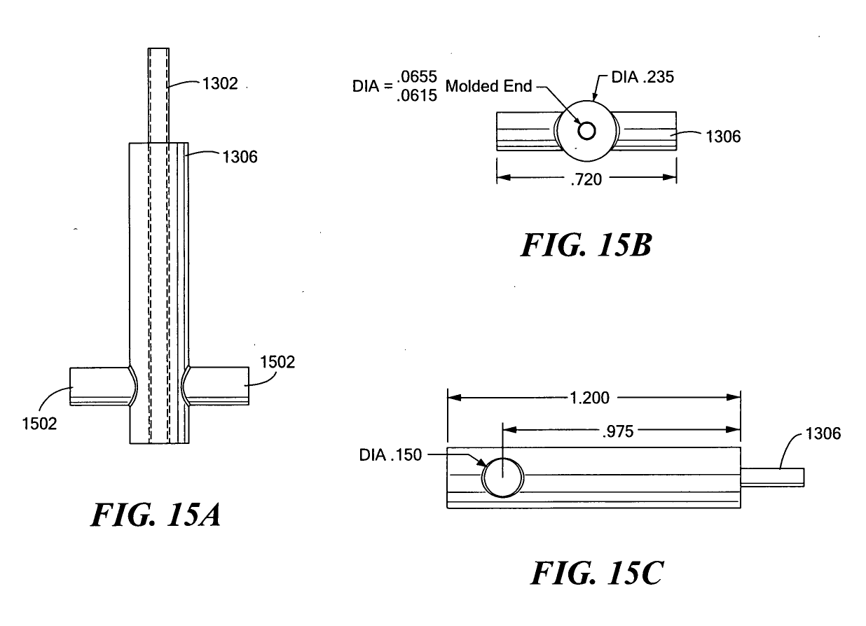

FIGS. 15A-15C are schematic diagrams showing the inner tube housing 1306 in greater detail. FIG. 15A is a top view of the inner tube housing 1306. FIG. 15B is an end view of the inner tube housing 1306. FIG. 15C is a side view of the inner tube housing 1306. The inner tube housing 1306 is typically molded onto the proximal end of the inner tube 1302, although it could be produced in other ways. The inner tube housing 1306 includes protrusions 1502 (one on each side) approximately 0.150 inches in diameter. During use of the tissue sample needle, the protrusions 1502 may be used for, among other things, preventing backward displacement and rotation of the needle during a thrusting of the needle and subsequently rotating the inner tube 1302 relative to an outer tube during operation of the snare 1304, as described in detail below. It should be noted that the overall dimensions of the inner tube housing 1306 may be substantially the same as the dimensions of the inner tube housing 106 so that either can be used in a common tissue sample needle actuator, as discussed below.

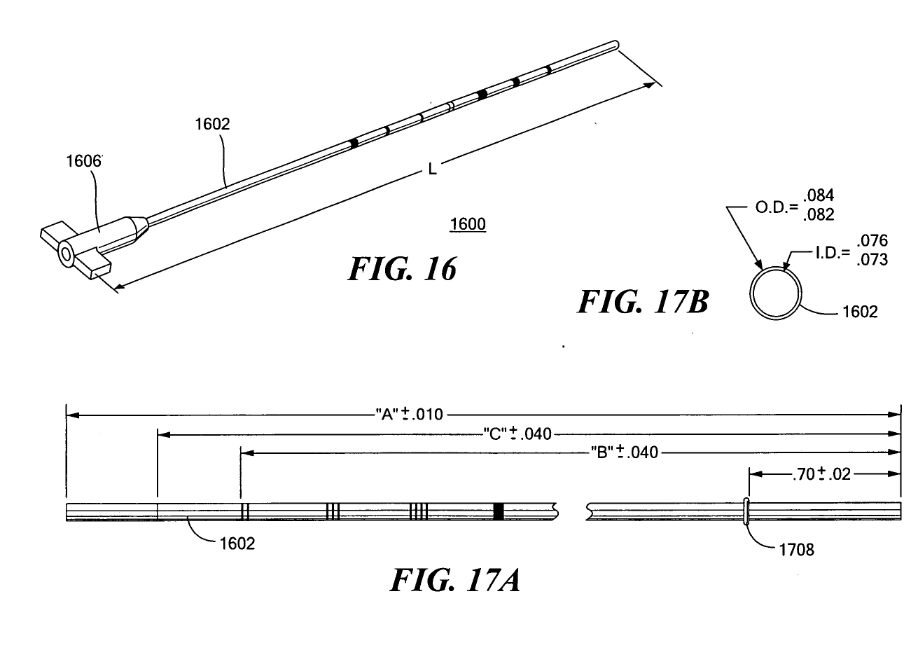

FIG. 16 is a schematic diagram showing an outer tube assembly 1600 for a 14-gauge tissue sample needle, in accordance with an exemplary embodiment of the present invention. The outer tube assembly 1600 includes an outer tube 1602 with an outer tube housing 1606 located at a proximal end. The outer tube 1602 typically includes depth indicator markings toward the distal end to aid in obtaining a tissue sample.

FIGS. 17A-17B are schematic diagrams showing the outer tube 1602 in greater detail. FIG. 17A is a side view of the outer tube 1602 showing the depth indicator markings at approximately one centimeter intervals along a length of the outer tube 1602 and also showing a wire ring 1708 that is formed on the outer tube 1602 approximately 0.70 inches from the proximal end to help secure the outer tube housing 1606 on the outer tube 1602. FIG. 17B is cross-sectional view of the outer tube 1602 showing outer diameter between approximately 0.082 and 0.084 inches and an inner diameter between approximately 0.073 and 0.076 inches.

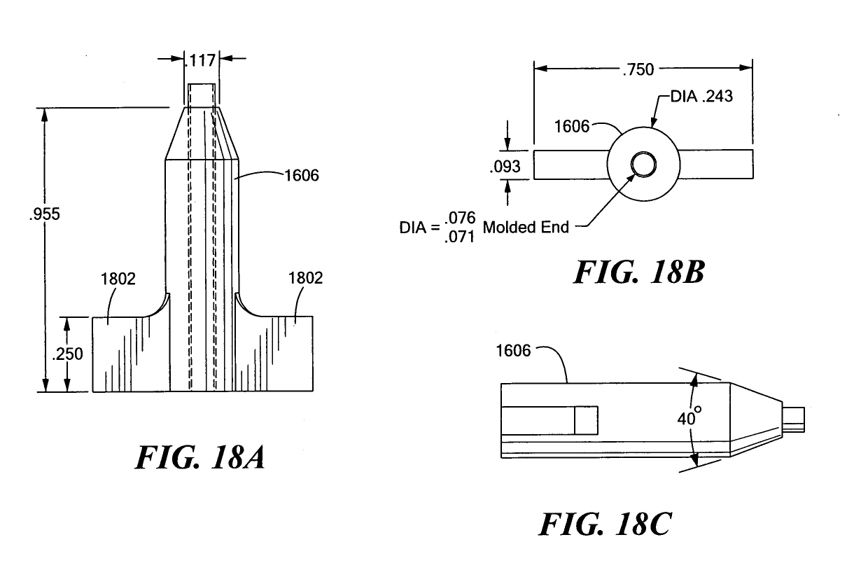

FIGS. 18A-18C are schematic diagrams showing the outer tube housing 1606 in greater detail. FIG. 18A is a top view of the outer tube housing 1606. FIG. 18B is an end view of the outer tube housing 1606. FIG. 18C is a side view of the outer tube housing 1606. The outer tube housing 1606 is typically molded onto the proximal end of the outer tube 1602, although it could be produced in other ways. The outer tube housing 1606 includes protrusions 1802 (one on each side). During use of the tissue sample needle, the protrusions 1802 may be used for, among other things, maintaining linear movement of the needle during a thrusting of the needle and preventing rotation of the outer tube 1602 during operation of the snare 1304, as described in detail below. It should be noted that the overall dimensions of the outer tube housing 1606 may be substantially the same as the dimensions of the outer tube housing 406 so that either can be used in a common tissue sample needle actuator, as discussed below.

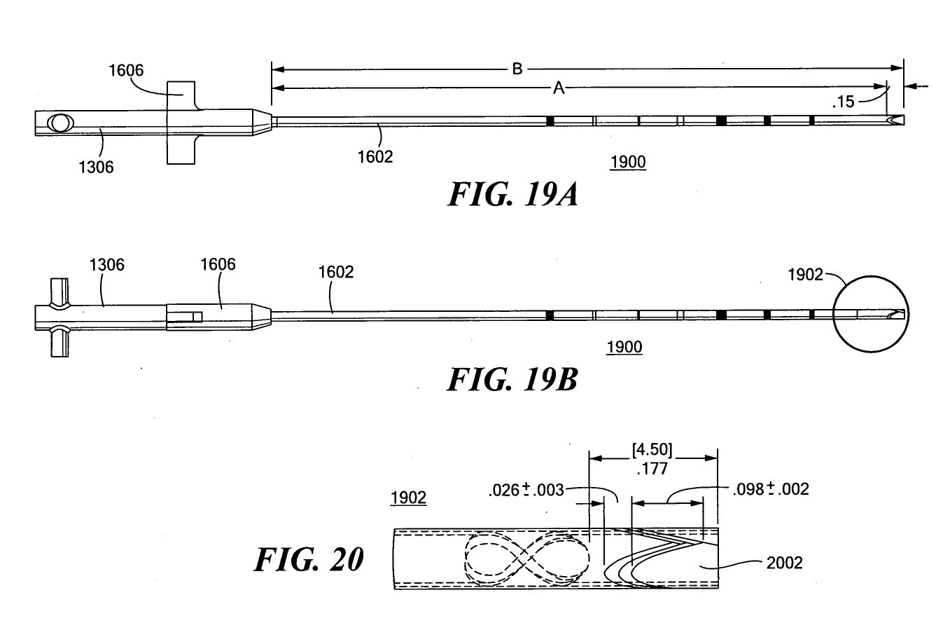

FIGS. 19A-19B are schematic diagrams showing a needle assembly 1900 in accordance with an exemplary embodiment of the present invention. The needle assembly 1900 includes an inner tube assembly 1300 disposed within an outer tube assembly 1600. The distal end of the inner tube 1302 (and, more particularly, the securing member 1408) is coupled to the distal end of the outer tube 1602, for example, by laser welding about a circumference of the distal end of the outer tube 1602. FIG. 19A is a side view of the needle assembly 1900 showing the inner tube housing 1306 protruding from the proximal end of the outer tube assembly 1600. FIG. 19B is a top view of the needle assembly 1900, highlighting the distal end 1902 of the needle assembly 1900 where the inner tube 1302 and outer tube 1602 are laser welded.

FIG. 20 is a schematic diagram showing the distal end 1902 of the needle assembly 1900 in greater detail. The inner tube 1302 and outer tube 1602 are laser welded in area 2002, which is in the area of the securing member 1408 and does not interfere with the deformable members 1404 and 1406. As discussed below, the distal end 1902 of the needle assembly 1900 is ground after welding in order to form a desired point configuration (e.g., a Franseen point). The weld is typically designed to substantially follow the outline of the grinding.

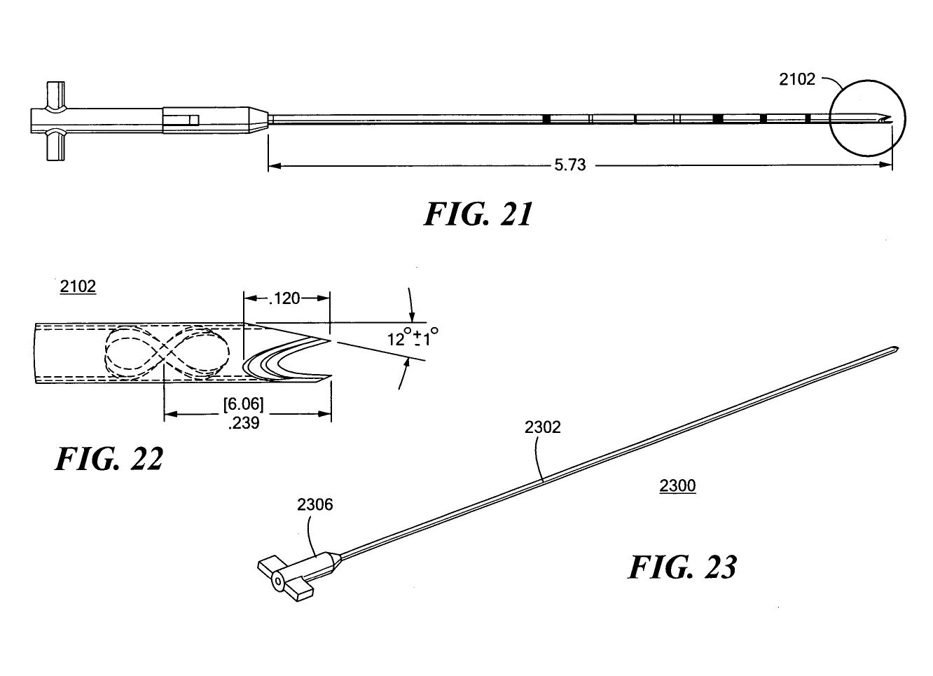

FIG. 21 is a schematic diagram showing the needle assembly 1900 after grinding. As mentioned above, the distal end 1902 of the needle assembly 1900 is ground after welding in order to form a desired point configuration 2102.

FIG. 22 is a schematic diagram showing the point configuration 2002 in greater detail.

FIG. 23 is a schematic diagram showing a stylet assembly 2300 for a 14-gauge tissue sample needle in accordance with an exemplary embodiment of the present invention. The stylet assembly 2300 includes a stylet 2302 and a stylet housing 2306. The stylet 2302 is typically sharpened at its distal end. The stylet assembly 2300 may be disposed within the inner tube assembly 1300 during operation of the tissue sample needle to facilitate insertion of the needle into a body tissue and/or to remove a tissue sample from the inner tube 1302. The stylet assembly 2300 is typically designed so that the sharpened tip protrudes from the distal end of the needle when the stylet assembly 2300 if fully inserted through the inner tube assembly 1300.

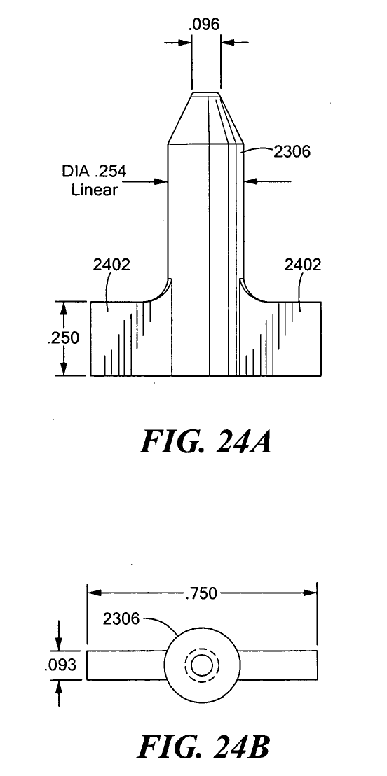

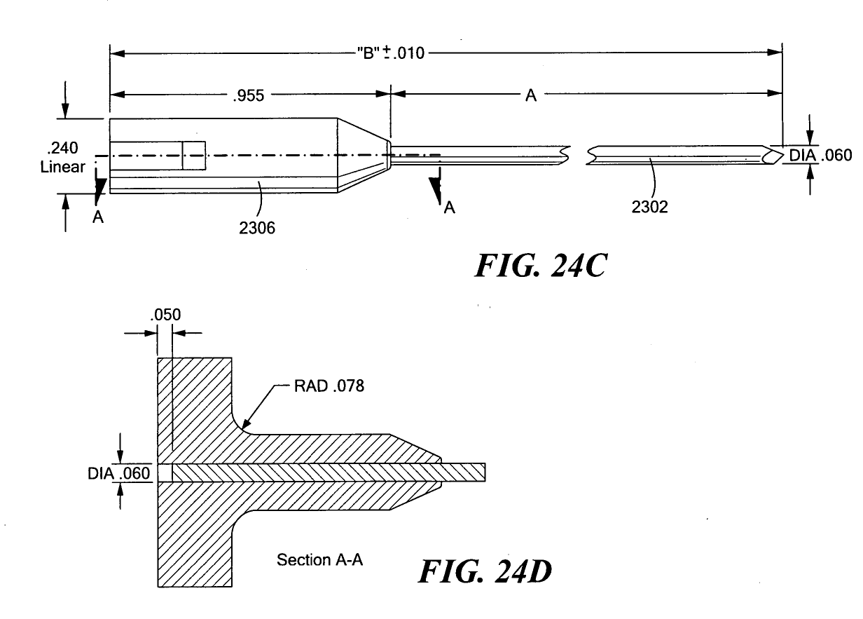

FIGS. 24A-24D are schematic diagrams showing the stylet housing 2306 in greater detail. FIG. 24A is a top view of the stylet housing 2306. FIG. 24B is an end view of the stylet housing 2306. FIG. 24C is a side view of the stylet housing 2306 showing the sharpened end. FIG. 24D is a cross-sectional view of the stylet housing 2306. The stylet housing 2306 is typically molded onto the proximal end of the stylet 2302, although it could be produced in other ways. The stylet housing 2306 includes protrusions 2402 (one on each side). During use of the tissue sample needle, the protrusions 2402 may be used for, among other things, securing the stylet assembly 2300, as described in detail below. It should be noted that the overall dimensions of the stylet housing 2306 may be substantially the same as the dimensions of the stylet housing 1106 so that either can be used in a common tissue sample needle actuator, as discussed below.

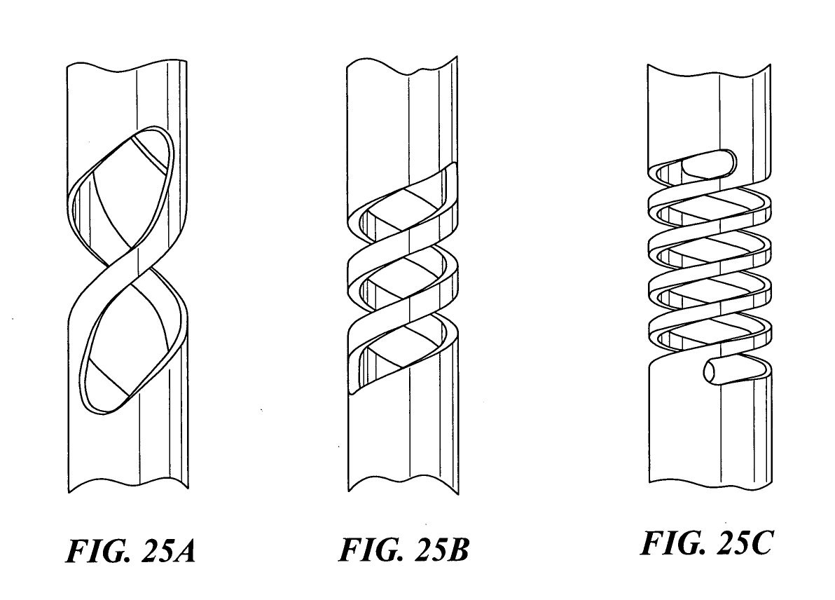

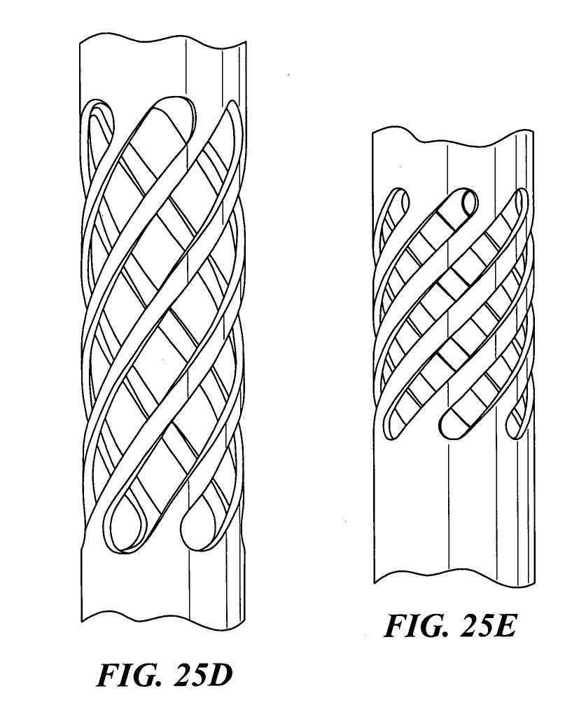

It should be understood that the present invention is not limited to any particular number, configuration, or placement of deformable members. In various alternative embodiments, the snare may include two, three, four, five, six, seven, or even more deformable members. FIGS. 25A-25E show some exemplary alternative snare configurations. FIG. 25A shows an enlarged view of an exemplary snare for a 14-gauge needle having two deformable members. FIG. 25B shows an alternative snare configuration with two deformable members, each forming a single coil. FIG. 25C shows an alternative snare configuration with two deformable members, each forming a double coil. FIG. 25D shows an alternative snare configuration with six deformable members. FIG. 25E shows an alternative snare configuration with seven deformable members. Of course, other snare configurations are possible.

It should also be understood that the present invention is not limited to any particular gauge or gauges of needles. Embodiments of the present invention can be made in 16-gauge, 18-gauge, 20-gauge, and other needle sizes. Different needle sizes may employ different snare configurations, including different numbers of deformable members and different configurations of deformable members. Even for a particular needle size, different types of needles, having different snare configurations, can be produced, for example, to meet specific requirements. For example, different types of body tissues may warrant different snare configurations.

It should be understood that the stylet assembly 1100/2300 is an optional component that can facilitate operation of the needle, for example, by facilitating insertion of the needle into a body tissue and/or facilitating ejection of a tissue sample from the needle, and should not be considered as a component of the needle itself. It should also be understood that the inner tube housing 106/1306 and the outer tube housing 406/1606 are used in certain embodiments of the invention for operation of the needle, and should not be considered as components of the needle itself.

In exemplary embodiments of the present invention, the inner tube housing 106/1306, the outer tube housing 406/1606, and the stylet assembly 1100/2300 are used during operation of the needle within an actuation system that includes an actuator, a needle assembly 700/1900, and a stylet assembly 1100/2300. The actuator includes, among other things, an actuator housing and an actuation mechanism disposed in the actuator housing for automatically thrusting the needle assembly 700/1900 forward and rotating the inner tube 102/1302 relative to the outer tube 402/1606 to operate the snare. The actuator also supports the stylet assembly 1100/2300 in such a way that the stylet 1102 protrudes from the needle when the actuator is in a cocked position to facilitate insertion of the needle into a body part, becomes recessed inside the inner tube 102/1302 when the actuator is fired (which thrusts the needle forward) to allow a tissue sample to be stored within the inner tube 102/1302, and ejects the tissue sample when the actuator is re-cocked (which withdraws the needle).

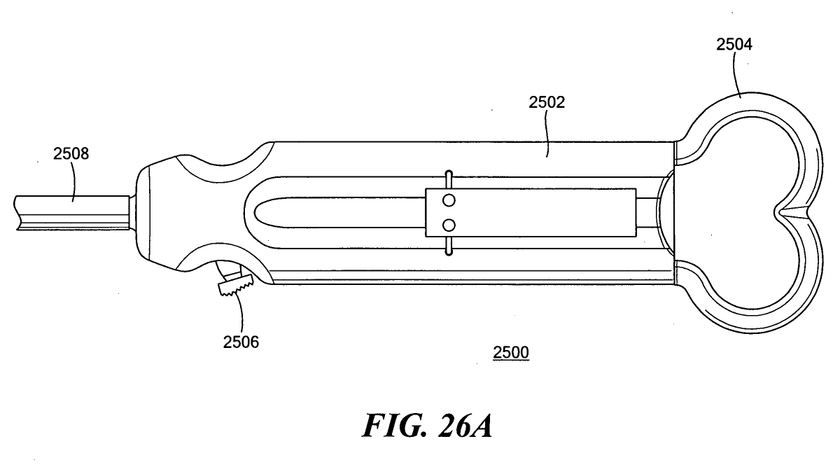

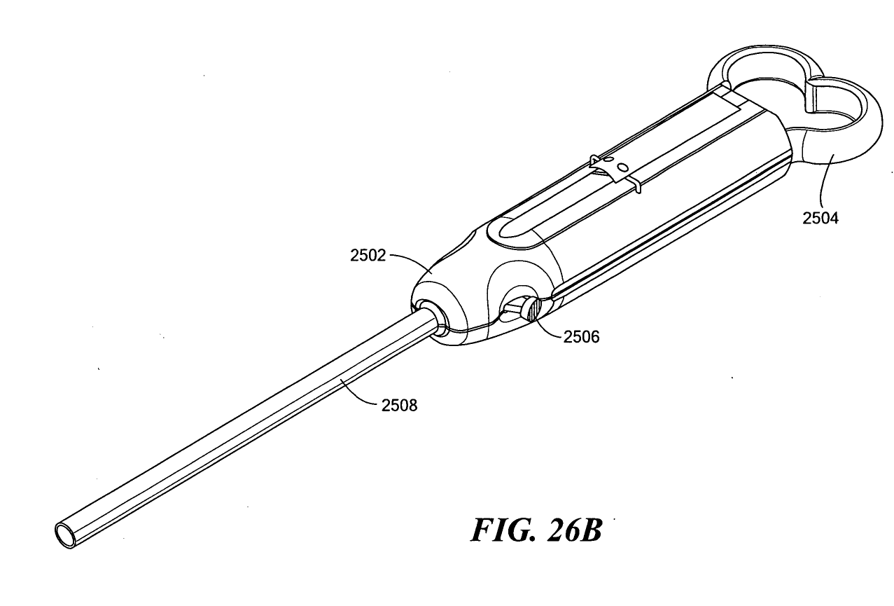

FIGS. 26A-26B are schematic diagrams showing an actuator 2500 in accordance with an exemplary embodiment of the present invention. FIG. 26A is a top view of the actuator 2500. FIG. 26B is a perspective view of the actuator 2500. The actuator includes, among other things, an actuator housing 2502, a handle 2504, a trigger 2506, and a removable protector 2508. The actuator housing 2502 is typically fabricated in two interlocking pieces. The handle 2504 is used to cock the actuator, specifically by pulling back on the handle 2504 while holding the actuator housing 2502. The trigger 2506 is used to fire the actuator to effectuate thrusting of the needle and operation of the snare.

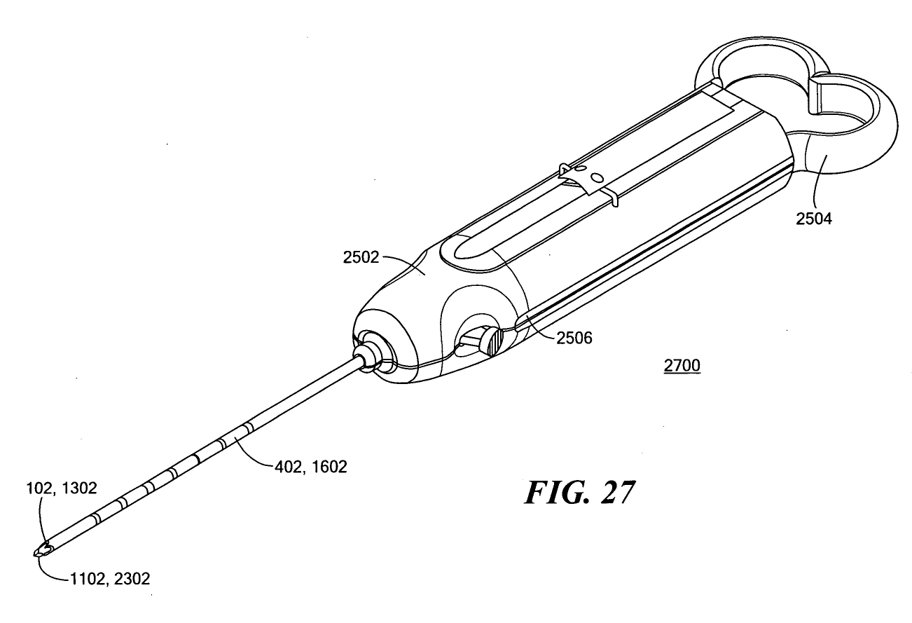

FIG. 27 is a schematic diagram showing an actuation system 2700 in accordance with an exemplary embodiment of the present invention. The actuation system 2700 includes an actuator 2500, a needle assembly 700/1900, and stylet assembly 1100/2300. The actuation system 2700 is shown here in a cocked position. The outer tube 402/1602 can be clearly seen, as can the sharpened tip of the stylet 1102/2302. The tip of the inner tube 102/1302 can be seen at the location where the inner tube 102/1302 and the outer tube 402/1602 are welded/ground. From the cocked position, depression of the trigger 2506 will cause the needle (including the outer tube 402/1602 and inner tube 102/1302) to be thrust forward through a predetermined distance (e.g., 25 centimeters) and subsequently the inner tube 102/1302 to be rotated relative to the outer tube 402/1602 through a predetermined range (e.g., 150 degrees) to operate the integral snare.

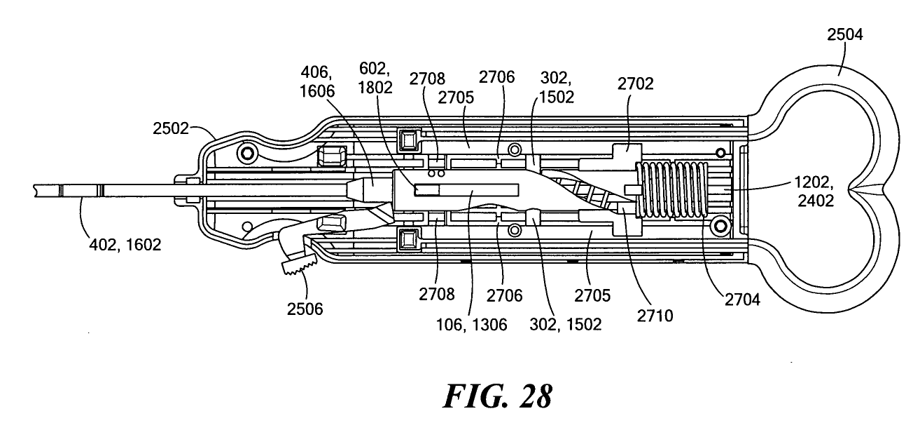

FIG. 28 is a schematic diagram showing a top view of the actuation system 2700 with the top cover of the actuator housing 2502 removed so as to expose the inner workings and components of the actuation system 2700. Again, the actuator system 2700 is shown in the cocked position. In this exemplary embodiment, a sleeve 2702, which houses the needle assembly 700/1900, controls both thrusting of the needle forward and subsequent rotation of the inner tube 102/1302 while securing the outer tube 402/1602. In this figure, a portion of the outer tube housing 406/1606 can be seen protruding from the front of the sleeve 2702, and one of the outer tube housing protrusions 602/1802 can be seen protruding through a slot in the top of the housing 2702. Also, a portion of the inner tube housing 106/1306 can be seen through the slot in the top of the housing 2702, and the inner tube housing protrusions 302/1502 can be seen protruding from the sides of the sleeve 2702. In the cocked position, the trigger 2506 prevents the sleeve 2702 from moving forward.

When the trigger is depressed so as to release the sleeve 2702, the sleeve 2702 is propelled forward through a substantially linear range of motion. During its forward motion, the sleeve 2702 does not rotate, but instead travels in a straight line along rails 2705. During a thrusting portion of the motion, the entire needle assembly 700/1900 is moved forward. During this thrusting portion of the motion, the inner tube housing protrusions 302/1502 ride along rails 2706 (which prevents both backward displacement of the needle assembly 700/1900 and rotation of the inner tube 102/1302) until the outer tube housing protrusions 602/1802 reach a stop within the actuator housing 2502 (stopping forward motion of the needle assembly 700/1900) and the inner tube housing protrusions 302/1502 reach a channel 2708. From this point, the sleeve 2702 continues to move forward through a snare operating portion of the motion in which the outer tube housing 406/1606 (and therefore the outer tube 402/1602 itself) is prevented from rotating by virtue of the outer tube housing protrusions 602/1802 being disposed within the slot in the top of the sleeve 2702 and the inner tube housing 106/1306 (and therefore the inner tube 102/1302 itself) is rotated by virtue of the inner tube housing protrusions 302/1502 riding within a serpentine or helical channel 2710 of the sleeve 2702. The channel 2710 effectively converts the linear motion of the sleeve to rotational motion of the inner tube 102/1302.

In this exemplary embodiment, the sleeve 2702 is propelled through its range of motion by a pair of springs 2704 that include an outer main spring that operates through the entire range of motion and an inner booster spring that effectively operates only through the thrusting portion of the motion in order to provide added force for thrusting the needle into dense or fibrous tissue. The springs 2704 are shown in a compressed state. The springs 2704 are compressed between the sleeve 2702 and the stylet housing protrusions 1202/2402.

In this exemplary embodiment, the stylet assembly 1100/2300 is held in a fixed position within the actuation system 2700. Specifically, the stylet 1102/2302 is disposed within the inner tube 102/1302, and the stylet housing protrusions 1202/2402 are disposed within fixed slots in the actuator housing 2502. When the needle is in the cocked position as shown, the sharpened tip of the stylet 1102/2302 protrudes from the end of the needle. After thrusting of the needle forward, the stylet 1102/2302 is effectively retracted into the inner needle 102/1302. When the needle is re-cocked, the needle is retracted so that the stylet 1102/2302 again protrudes from the end of the needle and any tissue sample stored in the inner tube 102/1302 is ejected.

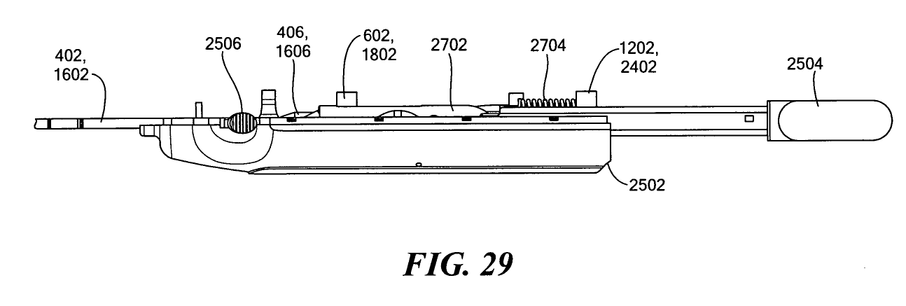

FIG. 29 is a schematic diagram showing a side view of the actuation system 2700. Various features, including the bottom of the actuator housing 2502, the handle 2504, the sleeve 2702, the outer needle 402/1602, the trigger 2506, a portion of the outer tube housing 406/1606 including an outer tube housing protrusion 602/1802, the springs 2704, and a stylet housing protrusion 1202/2402, can be seen.

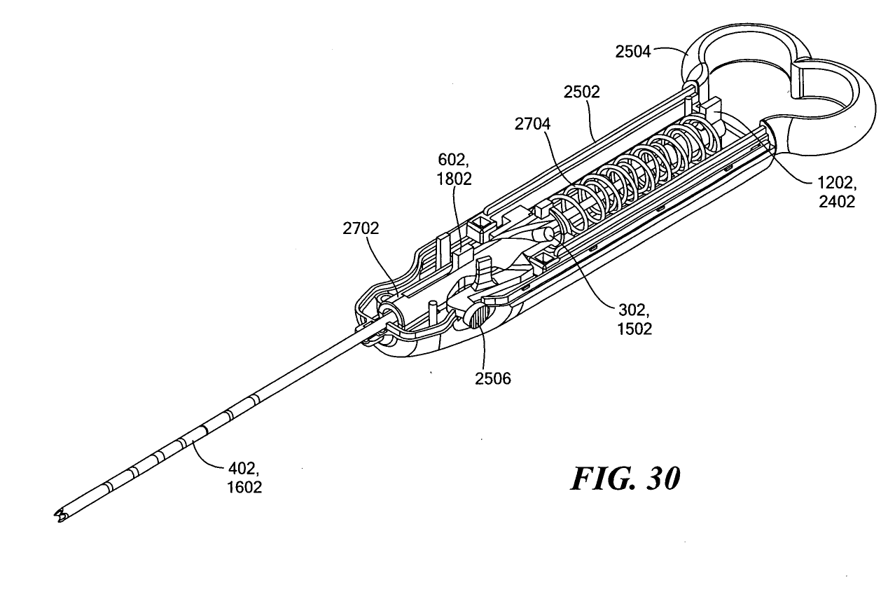

FIG. 30 is a schematic diagram showing a perspective view of the actuating system 2700 in a fired position. The sleeve 2702 has been released by the trigger 2506 and pushed forward through its complete range of motion by the springs 2704, which are now in a decompressed state. The needle has been thrust forward, as shown by the displacement of the outer tube housing protrusion 602/1802 relative to FIGS. 28 and 29, and the inner tube 102/1302 has been rotated relative to the outer tube 402/1602, as shown by the displacement of the inner tube housing protrusion 302/1502 relative to FIGS. 28 and 29.

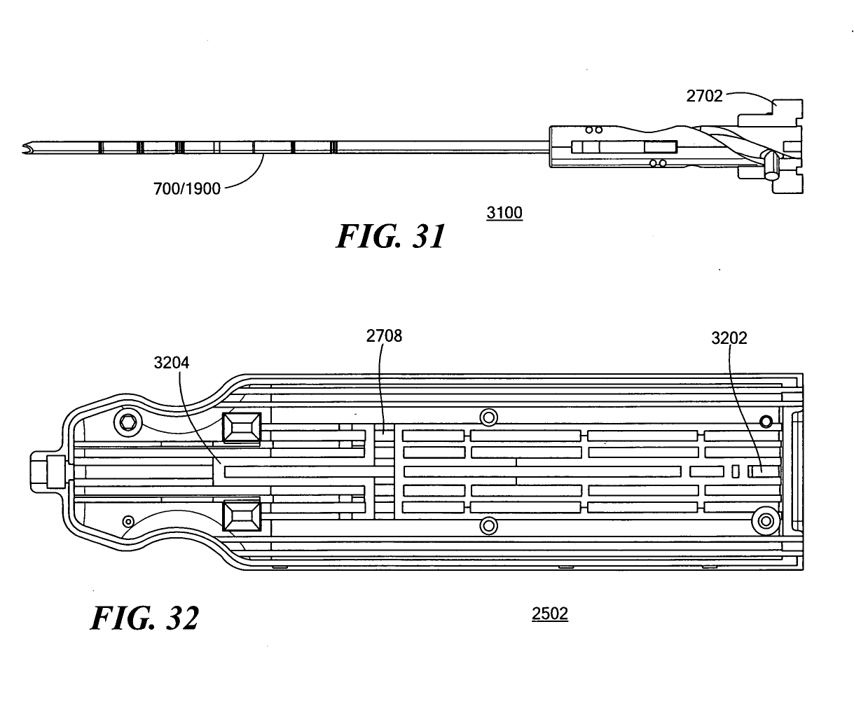

FIG. 31 is a schematic diagram showing the needle/sleeve assembly 3100 including needle assembly 700/1900 disposed within sleeve 2702. The needle/sleeve assembly 3100 is typically assembled and then introduced into the actuator housing 2502.

FIG. 32 is a schematic diagram showing a top view of the bottom portion of the actuator housing 2502. The actuator housing 2502 includes a fixed slot 3202 into which the stylet housing protrusion 1202/2402 is inserted. The actuator housing 2502 also includes the channel 2708 within which the inner tube housing protrusion 302/1502 is permitted to travel during rotation of the inner tube 102/1302. The actuator housing 2502 also includes a wall 3204 that is contacted by the outer tube housing protrusion 602/1802 to stop forward thrusting of the needle.

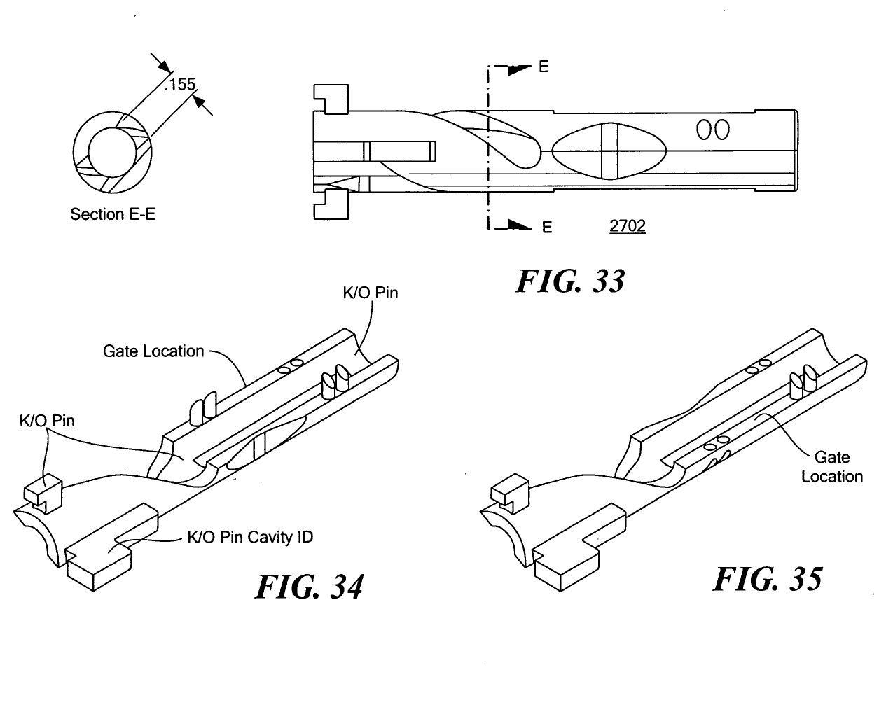

FIG. 33 is a schematic diagram showing a bottom view of the sleeve 2702. The sleeve 2702 is typically formed from two pieces of plastic. FIG. 34 shows one piece of the sleeve 2702. FIG. 35 shows the other piece of the sleeve. In order to form the needle/sleeve assembly 3100, the needle assembly 700/1900 is introduced into one piece of the sleeve 2702, and the other piece of the sleeve 2702 is then snapped into place. The stylet assembly 1100/2300 can be inserted through the inner tube 102/1302 of the needle assembly 700/1900 either before or after closure of the sleeve pieces.

It should be understood that, since the snare is operated by relative rotation of the inner and outer tubes, alternative embodiments of the invention could rotate the outer tube 402/1602 while constraining the inner tube 102/1302.

It should also be understood that alternative embodiments of the invention could perform just the thrusting of the needle or just the rotation of the inner tube. Alternative embodiments could also manipulate the needle in other ways such as, for example, rotating the entire needle (e.g., during thrusting, during operation of the snare, or during a separate phase), which may, in some situations, facilitate insertion of the needle and/or obtaining a tissue sample.

It should also be understood that thrusting and/or rotation could be driven by a mechanism other than one or more springs. For example, thrusting and/or rotation could be driven manually or by an electric motor, pneumatics, hydraulics, or other mechanism.

It should also be understood that cocking of the actuator could be powered rather than manual. For example, cocking of the actuator could be driven by an electric motor, pneumatics, hydraulics, or other mechanism.

It should also be understood that the present invention is not limited to a particular sleeve type or sleeve configuration or even to the use of a sleeve. Other mechanisms could be used to control thrusting and/or rotation, for example, various types of gears.

It should also be understood that the actuator is not limited to use with any particular type of tissue sample needle or snare configuration, and so can be used with single-coil snares of the type discussed in the background above, with multiple-member snares of the type discussed above with reference to FIGS. 1-24, or other types of snares.

It should also be understood that the actuator can be used without an integral stylet assembly 1100/2300. For example, a tissue sample could be removed from within the inner tube by manually inserting a stylet through either the proximal end of the needle or the distal end of the needle.

The present invention may be embodied in other specific forms without departing from the true scope of the invention. The described embodiments are to be considered in all respects only as illustrative and not restrictive.

What is claimed is:

1. An actuator for operating a tissue sample needle, the actuator comprising:
a housing; and
an actuation mechanism disposed within the housing for operating the tissue sample needle, the operating including at least automatically rotating an inner tube of the tissue sample needle relative to an outer tube of the tissue sample needle to operate an integral snare.

2. An actuator according to claim 1, wherein the operating further includes automatically thrusting the tissue sample needle prior to automatically rotating the inner tube relative to the outer tube.

3. An actuator according to claim 1, wherein the actuation mechanism rotates the inner tube while constraining the outer tube for automatically rotating the inner tube relative to the outer tube.

4. An actuator according to claim 1, wherein the actuation mechanism rotates the outer tube while constraining the inner tube for automatically rotating the inner tube relative to the outer tube.

5. An actuator according to claim 1, wherein the actuation mechanism comprises a movable sleeve for automatically rotating the inner tube relative to the outer tube.

6. An actuator according to claim 2, wherein the actuation mechanism comprises a movable sleeve for both automatically thrusting the tissue sample needle and automatically rotating the inner tube relative to the outer tube.

7. An actuator according to any of claims 5 or 6, wherein the tissue sample needle is disposed within the sleeve.

8. An actuator according to claim 7, wherein the actuation mechanism rotates the inner tube while constraining the outer tube for automatically rotating the inner tube relative to the outer tube, and wherein the sleeve includes a slot for constraining rotational movement of the outer tube and a channel for driving rotational movement of the inner tube during linear motion of the sleeve.

9. An actuator according to any of claims 5 or 6, wherein movement of the sleeve is driven by at least one of:
a spring;
a plurality of springs;
an electric motor;
pneumatics; and
hydraulics.

10. An actuator according to claim 1, wherein the actuation mechanism includes a cocked position in which the needle is prepared for operating.

11. An actuator according to claim 10, wherein the actuation mechanism includes a cocking mechanism for placing the actuation mechanism in the cocked position.

12. An actuator according to claim 11, wherein the cocking mechanism includes a handle for manually cocking the actuation mechanism.

13. An actuator according to claim 11, wherein the cocking mechanism is driven by at least one of:
an electric motor;
pneumatics; and
hydraulics.

14. An actuator according to claim 10, further comprising a trigger for selectively maintaining the actuation mechanism in the cocked position and releasing the actuation mechanism from the cocked position.

15. An actuator according to claim 2, wherein automatically thrusting the needle is performed in a first phase of actuation and automatically rotating the inner tube relative to the outer tube is performed in a second subsequent phase of actuation.

16. An actuator according to claim 15, wherein at least one of the housing and the actuation mechanism includes a stopping mechanism for stopping thrusting of the needle while allowing the subsequent rotation of the inner tube relative to the outer tube.

17. An actuator according to claim 2, wherein the housing is configured to hold a stylet such that the stylet protrudes from a distal end of the needle prior to thrusting and retracts from the distal end of the needle during thrusting.

18. A tissue sample actuation system comprising:
a tissue sample needle actuator; and
a tissue sample needle installed in the actuator, wherein the tissue sample needle includes an outer tube and an inner tube having an integral snare such that rotation of the inner tube relative to the outer tube operates the snare, and wherein the actuator includes an actuation mechanism disposed within a housing for operating the tissue sample needle, the operating including at least automatically rotating the inner tube relative to the outer tube to operate the integral snare.

19. An actuation system according to claim 18, wherein the operating further includes automatically thrusting the tissue sample needle prior to automatically rotating the inner tube relative to the outer tube.

20. An actuation system according to claim 18, wherein the actuation mechanism rotates the inner tube while constraining the outer tube for automatically rotating the inner tube relative to the outer tube.

21. An actuation system according to claim 18, wherein the actuation mechanism rotates the outer tube while constraining the inner tube for automatically rotating the inner tube relative to the outer tube.

22. An actuation system according to claim 18, wherein the actuation mechanism comprises a movable sleeve for automatically rotating the inner tube relative to the outer tube.

23. An actuation system according to claim 19, wherein the actuation mechanism comprises a movable sleeve for both automatically thrusting the tissue sample needle and automatically rotating the inner tube relative to the outer tube.

24. An actuation system according to any of claims 22 or 23, wherein the tissue sample needle is disposed within the sleeve.

25. An actuation system according to claim 24, wherein the actuation mechanism rotates the inner tube while constraining the outer tube for automatically rotating the inner tube relative to the outer tube, and wherein the sleeve includes a slot for constraining rotational movement of the outer tube and a channel for driving rotational movement of the inner tube during linear motion of the sleeve.

26. An actuation system according to any of claims 22 or 23, wherein the actuation mechanism includes at least one of a spring, a plurality of springs, an electric motor, pneumatics, and hydraulics for driving movement of the sleeve.

27. An actuation system according to claim 18, wherein the actuation mechanism includes a cocked position in which the needle is prepared for operating.

28. An actuation system according to claim 27, wherein the actuation mechanism includes a cocking mechanism for placing the actuation mechanism in the cocked position.

29. An actuation system according to claim 28, wherein the cocking mechanism includes a handle for manually cocking the actuation mechanism.

30. An actuation system according to claim 28, wherein the actuation mechanism includes at least one of an electric motor, pneumatics, and hydraulics for driving the cocking mechanism.

31. An actuation system according to claim 27, further comprising a trigger for selectively maintaining the actuation mechanism in the cocked position and releasing the actuation mechanism from the cocked position.

32. An actuation system according to claim 19, wherein automatically thrusting the needle is performed in a first phase of actuation and automatically rotating the inner tube relative to the outer tube is performed in a second subsequent phase of actuation.

33. An actuation system according to claim 32, wherein at least one of the housing and the actuation mechanism includes a stopping mechanism for stopping thrusting of the needle while allowing the subsequent rotation of the inner tube relative to the outer tube.

34. An actuation system according to claim 19, further comprising a stylet disposed within the actuator such that the stylet protrudes from a distal end of the needle prior to thrusting and retracts from the distal end of the needle during thrusting.

35. A method for obtaining a tissue sample, the method comprising:
cocking a tissue sample needle actuation system including a tissue sample needle installed in a tissue sample actuator;
inserting the tissue sample needle into a body part; and
firing the tissue sample needle actuation system to operate the tissue sample needle, the operation including at least automatically rotating an inner tube of the tissue sample needle relative to an outer tube of the tissue sample needle to operate an integral snare.

36. A method according to claim 35, wherein the operating further includes automatically thrusting the tissue sample needle prior to automatically rotating the inner tube relative to the outer tube.

37. A method according to claim 35, wherein the actuator rotates the inner tube while constraining the outer tube for automatically rotating the inner tube relative to the outer tube.

38. A method according to claim 35, wherein the actuator rotates the outer tube while constraining the inner tube for automatically rotating the inner tube relative to the outer tube.

39. A method according to claim 35, further comprising:
withdrawing the tissue sample needle from the body part; and
ejecting a tissue sample from the tissue sample needle.
